(19) United States
(12) Patent Application Publication
(10) Pub. No.: US 2012/0156675 A1
(43) Pub. Date: Jun. 21, 2012

(54) PICOWELL CAPTURE DEVICES FOR ANALYSING SINGLE CELLS OR OTHER PARTICLES

(75) Inventors: Dietrich Wilhelm Karl Lueerssen, Oxford (GB); Daniele Malleo, Oxford (GB)

(73) Assignee: OXFORD GENE TECHNOLOGY IP LIMITED, Oxford (GB)

(21) Appl. No.: 13/377,211

(22) PCT Filed: Jun. 9, 2010
(86) PCT No.: PCT/GB2010/001133

§ 371 Date: Feb. 27, 2012

(30) Foreign Application Priority Data
Jun. 9, 2009 (GB) 0909923.5

(51) Int. Cl. G01N 33/53; B29C 41/42; C12M 1/00; B29C 33/42; C12M 1/34; C12Q 1/02

(52) U.S. Cl. 435/6.11; 435/287.1; 435/29; 435/287.2; 435/283.1; 249/117; 264/334

(57) ABSTRACT

A convenient way of isolating individual cells permits individual analysis of their contents. A capture support for individually capturing cells of interest comprises a first surface including at least one well sized to accommodate an individual cell, wherein the support is made of a differentially permeable material which permits transfer of a solvent and any low molecular weight species through the support from a second surface of the support to a well, but which is substantially impermeable to biopolymers. Single cells are captured, their contents are released, and the contents of individual cells are then analysed within a chamber containing suitable analytical components e.g. immobilised nucleic acid probes, immobilised antibodies, etc. Analysis of a single cell's genome, transcriptome, proteome, etc. thus becomes possible.

### TECHNICAL FIELD

This invention is in the field of cell analysis, and in particular the analysis of individual cells.

### BACKGROUND ART

There are many methods for biochemical characterisation of cells and tissues. Methods such as electrophoresis, chromatography, mass spectrometry, microarrays, etc. are used to analyse the molecular composition of cells or tissues. The results of such analyses may indicate a disease state, for example. Analyses are most often carried out after lysing cells to release their contents, and it is usually necessary to use a large number of cells, because it is difficult to isolate single cells and because normal methods of detection are not sensitive enough to measure the contents of single cells.

It is rare, however, to find a living system comprising cells that are all in the same state: cell cultures artificially synchronised in the laboratory may approach homogeneity, but cells even of the same type in a natural situation will be in different states e.g. at different stages in the cell cycle, etc. Typical analyses thus represent an average of cells being analysed.

For a more complete description of the state of any system, it would be advantageous to analyse individual cells. For example, many disease states in humans elicit changes to the white blood cells, and in Hodgkin's lymphoma it has been shown that the gene expression pattern of individual lymphocytes is not representative of the population as a whole [1]. Analysis of a mixture of cells thus masks heterogeneity within the mixture, and fails to provide information which is likely to be important for understanding the disease state. Subtle but important variations between cells are lost due to the inadequacies of such a method.

There are many examples in biology and medicine where analysis of individual cells would be more useful than analysis of a whole population or collection. It is a major objective of developmental biology to have a description of the molecular changes that accompany growth and differentiation of an organism.

Reference 2 coined the term “chemical cytometry” to describe the use of high-sensitivity chemical analysis techniques to study single cells, and reference 3 reviews basic features of single cell analysis. Reference 4 reviews microtechnologies and nanotechnologies for single-cell analyses. Reference 5 describes microfluidic devices for manipulating single cells. Single cell isolation apparatuses are disclosed in references 6, 7 & 8.

It is an object of the invention to provide further and improved devices and processes for analysing individual cells, and in particular their genomes, transcriptomes and proteomes.

### DISCLOSURE OF THE INVENTION

The invention provides in general a convenient way of isolating individual cells in an apparatus which permits individual analysis of their contents. Single cells are captured, their contents are released, and the contents of individual cells are then analysed within a chamber containing suitable analytical components e.g. immobilised nucleic acid probes, immobilised antibodies, etc. Analysis of a single cell's genome, transcriptome, proteome, etc. thus becomes possible. Moreover, by arranging multiple chambers in the same device, multiple cells can simultaneously be treated and analysed in parallel, allowing individual cells within a population to be compared rapidly and conveniently.

The invention provides a capture support for individually capturing cells of interest, comprising a first surface including at least one well sized to accommodate an individual cell, wherein the support is made of a differentially permeable material which permits movement of a solvent and any low molecular weight species through the support from a second surface of the support to a well, but which is substantially impermeable to biopolymers.

In use, a cell is applied to the capture support and is caught in a well. The cell enters the well intact, and remains intact while the support is joined to a lid, closing the well, to form a chamber. Once within the chamber, the cell cannot exit. The cell is then lysed within the chamber, and the released cellular biopolymers are kept within the chamber.

Thus the invention provides a device for analysing the contents of an individual cell comprising a capture support and a lid for receiving the contents of a well.

In a preferred embodiment the capture support has a plurality of wells, such that the contents of multiple cells can be separately analysed in parallel after the support is joined to a lid which seals the plurality of wells. Performing identical individual analysis in parallel on different cells is particularly powerful and readily allows differences to be detected in nominally identical cells. In another embodiment, the analysis performed differs between individual cells, by allowing the contents of the individual cells to interact with differing sets of analytical components.

Although single cell capture is a preferred way of using the devices, other analyses are also possible. For example, capture of more than a single cell in a single well may be desired, such as the capture cell spheroids or blastocysts. Preferably fewer than 10 cells will be captured in a single well, for example 9, 8, 7, 6, 5, 4, 3, or 2 cells.

Other non-cellular particles can also be analysed. Thus the invention also provides a capture support for individually capturing particles, comprising a first surface including at least one well with a diameter of less than 200 μm and depth of less than 200 μm, for example diameter and depth less than 150 μm, less than 100 μm, less than 50 μm, less than 40 μm, less than 30 μm, less than 20 μm, or less than 10 μm, wherein the support is made of a differentially permeable material which permits movement of a solvent and any low molecular weight species through the support from a second surface of the support to a well, but which is substantially impermeable to biopolymers. The particles to be caught will typically be cells, organelles (for example mitochondria, chloroplasts, etc.), liposomes, viruses or functionalised beads. Accordingly, many features and methods described below for capture and analysis of cells in capture supports are also appropriate for capture and analysis of particles in a capture support. In one embodiment the capture support can capture a plurality of particles in a single well. This plurality may comprise a number of particles of the same type, for example a group of cells. In an alternative, it is capable of capturing particles of different types, for example a cell and a functionalised bead in a single well. In this case the bead may then be subjected to downstream analysis.

The capture support is capable of locating the particles at a known position, i.e. the positions of the wells in the first surface of the capture support. The capture support provides a known spatial arrangement that permits the results of any downstream analysis.

The invention provides a process for analysing one or more particles (e.g. cells), comprising steps of: capturing the particle(s) within well(s) on a surface of a capture support as defined above; sealing the well(s) with a lid, capable of sealing the well(s), to form one or more chamber(s) from which captured cells cannot exit; releasing the contents of cell(s) such that they remain in the one or more chambers; allowing the released contents to interact with one or more analytical component(s) within the one or more chambers, thereby permitting analysis of the contents.

Different analyses can require different devices within the scope of the invention. For instance, different particles (e.g. cell types) may require devices with different dimensions. Different analyses of the same cell type may use different analytical components e.g. for proteome analysis vs. transcriptome analysis, or for cell cycle analysis vs. cell signalling analysis. Further, as described in more detail below, devices which analyse only one cellular component within a single chamber may have different dimensions and/or design, and a different arrangement of analytical components in comparison to devices which analyse a variety of cellular components within a single chamber.

Moreover, devices can be designed based on previous experimental data, and can be used in different ways depending on previous experience. For example, if a device fails to give useful data in an initial experiment, variables such as capture support material, temperature of operation, the type of analytical component, dimensions, etc. can be altered in further experiments. Different experiments can thus use different features, as described herein, depending on the desired analysis. Alternatively, numerical or analytical simulations can be carried out in order to optimise the design features. As an example, random walk simulations are well known to evaluate diffusion of molecules, which can be used to evaluate different features on the performance of the device.

Capture supports of the invention can be made in various ways. In one method, the material from which the support is made can be cast in a mould. The mould shape is the inverse, or negative, of the desired capture support. Design of the mould allows capture supports described above to be manufactured. Thus the invention provides a mould for the manufacture of a capture support according to the invention, wherein the mould is capable of receiving material which solidifies within the mould.

The invention also provides a method for the manufacture of a capture supports, said method comprising the steps of:
a) adding to a capture support mould a material capable of solidifying;
b) incubating the mould under conditions which permit the material to solidify; and
c) separating the mould and the solidified material.

Capture Supports

The capture support is made from a material which permits a solvent and low molecular weight solutes to travel through the support, but is impermeable to biopolymers of interest, and comprises one or more wells on the surface of the support.

Usually, the cell capture support will be sized to fit standard equipment. For instance, the capture support may have dimensions of a similar order to a microscope slide or a 96-well microtitre plate. The capture support may also be functionalised, so that its wells contain reagents employed in the downstream processes.

Wells

The capture support includes one or more wells. A well is preferably arranged such that it can capture only a single cell. This will typically be achieved by ensuring that the dimensions are such that the well can accommodate only a single cell of interest at any time. The wells in the surface of the support are pits or holes or indentations in the surface of the capture support. The well is an indentation which is exposed to the environment of the capture support during use. This exposure permits a particle to enter the well where it can be captured. The well can be sealed, and when this sealing occurs (e.g. by the addition of a lid) a chamber is formed, such that the particle cannot exit the chamber intact. Typically the wells are made by appropriate design of a cast to exclude material from the region desired to be a well. In an alternative, a well may be made by excision of surface material, for example by laser ablation. Further methods include wet chemical etching (isotropic and anisotropic), electrochemical etching, wet photoetching, dry chemical etching, sputter etching, ion milling, reactive ion etching and deep reactive ion etching (DRIE), x-ray deep-etch lithography and electroforming (a.k.a. LIGA), vapour phase etching (with XeF₂), focused ion beam milling, laser machining, ultrasonic drilling, electrical discharge machining, mechanical drilling, milling, grinding, honing, lapping, sawing; hot embossing, injection moulding, microthermoforming and casting (see reference 9 for a review).

Further, at least a portion of a well's surface may be coated with reagents which aid capture of particles such as cells, for example antibodies to a specific cell surface marker. The capture reagent within each well may be the same. In an alternative, different capture reagents may be located in different wells in a capture support. In doing so, different wells in a plurality of wells may be adapted such that they are capable of capturing different particle types. Such adaptation may be useful for capturing different cells in a cell suspension which naturally contains a plurality of cell types, for example blood or lymph fluid. This adaptation may be used to complement any differences in well dimensions employed to capture different cell types. In doing so, use of more shallow wells may be possible.

Routinely the wells have a substantially cylindrical shape, with a flat base parallel to the surface and with vertical sides. Other shapes of wells can also be made in the surface of the capture support. A cylindrical well has circular cross-section. Included in the scope of the invention are wells with cross-sections of other shapes of N sides, where N may be 1, 2, 3, 4, 5, 6 etc. The wells may contain stepped increases in diameter from the base of the well to the surface of the capture support. The base of the well may also be oriented so that it is not parallel to the surface. The base of the well may be curved. The wells may be conical, with the broad base of the cone open at the surface of the capture support.

A capture support will typically contain a plurality of wells. For example, the number of wells included in a capture support may be more than 10, more than 50, more than 100, more 25 than 500, more than 1,000, more than 5,000, more than 10,000, more than 50,000, more than 100,000, more than 500,000 or more than 1,000,000.

In a capture support comprising a plurality of wells, the wells will preferably have substantially identical dimensions. In applications where a range of different cell types are to be caught by a single capture support, then wells of varying dimension may be employed.

Preferably the plurality of wells will be arranged in a repeating regular array. The wells may be arranged in rows and columns, and the rows may be perpendicular to the columns (e.g. see FIG. 4). In an alternative, the wells may be packed more tightly, for example in hexagonal arrays.

Design of the dimensions of the wells can ensure single occupancy of each well. The wells need to be sized appropriately for the particle to be analysed. Appropriate dimension of the capture support are discussed further below.

When the lid is joined to the capture support the particle is sealed within a cylindrical chamber, made from the well and lid, where analysis takes place. A cross-sectional view of the capture support comprising wells is shown in FIG. 3, whilst FIG. 4 is an image of cells captured in such wells. Centre-to-centre spacing can be varied depending on how dense the desired arrays need to be. In any case, F>C.

A well may be surrounded by a recess in the surface of the capture support, such that, when sealed by the lid, the chamber formed includes both the well and the recess. FIG. 5 shows a well of this type, whilst FIG. 6 is an image of cells captured in such wells. Whilst FIG. 5 shows a circular recess, other shapes of recess may be employed, for example wells of N sides as detailed above. Whilst FIG. 5 shows a recess concentric to the well, this arrangement is not necessary. A well's footprint when sealed will be the same as the well's cross-sectional area, but addition of a recess means that the footprint also includes the recess.

When a well surrounded by a recess is sealed by a lid, the area of the lid contained within the chamber formed is the same as the cross-sectional area of the recess. Accordingly, in a chamber formed from a lid and a well with a recess, a larger area of the lid will be contained in the chamber in comparison to a chamber formed from that well without a recess. A cross-section through a well of this form is shown in FIG. 5. In this figure, dimensions B and C determine the size of the well. Dimension A determines the footprint of the well on the lid. Dimension D determines the depth of the recess. As described above, the centre-to-centre spacing of wells can be varied depending on how dense the desired arrays need to be. Here, the centre-to-centre spacing should be greater than the diameter of the footprint (A in FIG. 5).

As noted above, the centre-to-centre spacing of the wells included in a capture support will vary depending upon the dimensions of the wells and the desired array density. For example, the centre-to-centre distance may be less than 500 μm, less than 300 μm, less than 250 μm, less than 200 μm, less than 150 μm, less than 100 μm, less than 50 μm, less than 40 μm, less than 30 μm, or less than 20 μm, but will always be greater than the well diameter.

Though depicted in FIG. 5, the base of the recess in the capture support does not need to be parallel to the surface of the capture support. For example, the recess may be angled to form a shelf with a single surface connecting the surface of the capture support to the wall of the well. Similarly, the profile of the recess may be curved, such as a concave surface or a convex surface. Other variations will be evident.

In one embodiment, at least a portion of the surfaces of a well on the capture support may be coated with one or more analytical reagents which permit analysis of biopolymers after their release in the chamber. The analytical reagents may be located on the base of the well, on the side of the well, or on the surface of the recess surrounding the well. The surface of the recess may contain an array of analytical components. Potential analytical components which may be located on the surface of the wells are discussed below. Methods of linking oligonucleotides to functionalised polyacrylamide supports are detailed in reference 10.

The well may contain treatment reagents, for example lyophilised enzymes, enzymes which become active upon heating, or reagent-containing liposomes which can be lysed upon demand at a different stage of the experiment, for example by lasers or by the application of detergent solutions. The time of lysis of any liposome can be tailored to be suitable to the experiment being performed. For example, liposomes may be lysed to release reagents which then lyse cells, which are present in the chambers. As an alternative, the liposomes may be lysed at the same time as cell lysis. Further, the liposomes may be lysed following cell lysis, for example to release reagents appropriate to treatment of components released from the particles, such as DNases or proteases, or reagents for use in other downstream processes.

Materials

Suitable materials for the manufacture of the capture support are well known. The material used to make the capture support should be impermeable to the particle under analysis (e.g. a cell). The material should also be substantially impermeable to biopolymers released from the particle, or released upon lysis of a cell. These biopolymers may be RNA (including mRNA, rRNA and miRNA), DNA, polypeptides and/or polysaccharides. Although cellular components may be capable of slowly diffusing through the material of the capture support over extended time periods, for example over one or more days, this diffusion should be limited when the cellular and analytical components are interacting. The material should permit any solution of low molecular weight reagents applied to the surface of the capture support in solution, such that the reagents can travel through the support. This differential permeability ensures that the biopolymers are unable travel through the material upon lysis of the cell and so inter-well contamination (i.e. well-to-well leakage) cannot occur.

The limit of permeability may be altered by appropriate design of the material of the capture support. The capture support is thus tuneable to the precise experimental procedure employed. For example in a capture support which is an acrylamide gel, the % acrylamide in the capture support can be varied. A higher concentration of acrylamide will result in a support which has a lower cut off value of permeability, while a lower concentration of acrylamide permits molecules of a higher molecular weight to travel through the support. Thus a material may be chosen so, for example, small RNAs (e.g. miRNAs) can travel either through the support or be retained.

The support will permit the movement of low molecular weight species with MW of less than 1000 Da, but will be impermeable to captured particles, and mRNA. As mentioned above, the material can be tuned for the retention of different molecular species. In some embodiments, therefore, the limit for permeability may be higher than 1000 Da, for example 2000 Da, 3000 Da, 4000 Da, 5000 Da, 6000 Da, 7000 Da, 8000 Da, 9000 Da, 10000 Da, or more than 10000 Da. As the permeability of the support increases, some biopolymers may be allowed to travel through the support. For example in a support with a limit of permeability greater than ˜3500 Da, 30 mer peptides may travel through the support. Where the limit of permeability is greater than ˜6000 Da, 20 mer miRNAs may travel through the support. In a capture support with a limit of greater than ˜12500 Da, small proteins, such as cytochrome c may travel through the support. The limit of permeability can be chosen according to the biopolymers of interest.

Where a lysis reagent is used to lyse the cells captured in chambers formed from the wells of the capture support, the material should be permeable to this lysis reagent so that the lysis reagent can travel through the support from the surface where it is supplied to the wells. Similarly, if other reagents, such as low molecular weight analytical reagents or treatment reagents, for example dyes or fluorimetric or colorimetric substrates for assays, are applied to the surface of the capture support, the material should similarly be permeable to these reagents. Further, the materials should not be disruptive to analysis. The material should not interact with the particles, biopolymers or reagents in an undesired fashion, for example the components should not non-specifically bind to the materials. Further, unreacted compounds which remain in the support as artefacts of manufacture should not leach out of the material such that any processes of lysis and analysis are perturbed. Preferably the capture support is made from a permeable polymer, such as a polyacrylamide gel. In an alternative, another polymer which is made from modified acrylamides, for example from methylacrylamide monomers, may be used. Such modifications to the acrylamide skeleton can alter the properties of the material, for example in terms of their hydrophobicity/hydrophilicity or charge, or facilitate the subsequent attachment of reagent by increasing the number of functional moieties in the final product. Such modifications may be preferable depending upon the application of the device to ensure cells, cellular components or reagents do not adhere to the capture support a manner which is disruptive to the analysis. Other materials may also be used, provided that they are suitably permeable to any reagents used during lysis and analysis. Typical materials comprise hydrophilic and liquid permeable polymers inclusive of agarose and polyacrylamide gels, poly(vinyl acetate), polystyrene, poly(vinyl carbazole), poly(methyl methacrylate), polyisobutilene, polyacrylates, polyisoprene, polybutadiene copolymers or combinations thereof or water permeable silicone gels.

Use may also be made of fibres formed from polyvinyl chloride, Teflon or other fluoropolymers, polysulfone, nylon, polycarbonate, polyvinylidene fluoride, polyamide, polyester cellulose acetate and, nitrocellulose, though preferably non-fibrous materials are used.

Dimensions

The capture support should be capable of receiving one or more lids. Increasing the size of the device has both advantages and disadvantages. When made from a polymer such as acrylamide, thin capture supports will be fragile and prone to tearing. An increase in size may increase the distance between the first surface where the wells are located, and second surface where the liquid which travels through the support is applied. An increase in distance will correspond to an increase in time taken for the liquid to move through the support. Ideally the capture support will be 500-1000 μm deep. The depth across which the liquid must move, and accordingly the time taken, frequently important experimental consequences. For example, in use, if a cell is still metabolically active, the longer that the cell is held within the well before lysis provides a larger window for any cellular enzymes, for example RNases or proteases, to degrade the cellular components of interest. Therefore, the thickness of the capture support determines the time taken between capture and lysis in embodiments where a lysis reagent is employed. In such a case, a thin support is preferred as this minimises the time taken for any lysis reagent to travel through the support. While a thinner capture support will allow faster travel of reagents through its structure, a balance must be struck because the thinner capture support is, the weaker it is and so the more likely it will be to break in usage.

In some embodiments, however, the impact of the thickness of the capture support may not be so relevant. As discussed in more detail below, the particles caught by the capture support may be arrested or fixed by the application of reagents (e.g. methanol), so that the cells are no longer metabolically active. By inactivation of the cellular enzymes, for example cellular RNases, DNases, proteases etc., the cellular biopolymers of interest are no longer so sensitive to the time taken from capture to lysis. In this case a longer time for the reagents to travel through the support may be tolerated and so a thicker, more robust capture support may be used.

The dimension of the wells in the capture support should be designed to be appropriate to the particles being caught. Using a capture support for capture of an individual mammalian cell (of diameter 10-15 μm) as an example, and with reference to FIG. 3, the depth is typically between 1.5-2 times that of the cell diameter (B=15-30 μm), while the well diameter is typically 1.2-1.333 times the cell diameter (C=12-20 μm).

Where capture of multiple particles are desired, then different multiples of the particle diameter may be employed, for example depth and diameter of 2, 3, 4, 5 or more times greater than the diameter of the particle to be caught.

A recess surrounding a well is ideally of a depth less that the diameter of the cell type being analysed, preferably less than half of the diameter of the particle being analysed. Preferably, the recess is between about 0.1-5 μm deep, for example preferably about 1-4 μm deep or 2-3 μm deep. The primary factor in design of the depth of the recess is that it is sufficiently shallow to ensure that it does not capture additional cells, and that any cells in the recess are easily removed by washing. The diameter of the recess can be significantly greater than that of the well, for example many hundreds of μm when the diameter of the well often will be in the order of tens of μm. Preferably, the diameter is about 50-500 μm.

An upper limit to the diameter of any recess is set by the time taken for cellular components to diffuse through the chamber following lysis. The relative dimensions of the recess (the diameter:depth ratio) should also be such that the recess does not collapse in on itself during use of the capture support, e.g. when inverted. This ratio will vary with strength of the material used to manufacture the capture support, but typically the diameter:depth ratio will be less than 10000:1, for example less than 5000:1, less than 1000:1, less than 500:1, or less than 100:1. Whilst a smaller diameter recess ensure more rapid diffusion of analytical components across is, a larger diameter recess permits more analytical components to be incorporated into a chamber.

The centre-to-centre distance of the wells (dimension F in FIG. 3) also requires careful consideration. The biopolymer contents of different chambers formed from the capture support and lid should not be in communication. By increasing the centre-to-centre dimension this can be ensured. The upper limit for this distance is imposed by a desire to provide the densest array of wells possible, such that in use the maximum possible number of cells is caught by the capture support. The centre-to-centre distance is closely related to the diameter of the well. When the diameter centre-to-centre distance, then the wells will be in communication. Therefore it is preferable that the centre-to-centre distance is larger than the well diameter, for example 2×, 5×, 10× or more than 10× the well diameter. In those wells containing recesses, the centre-to-centre distance should be larger than the diameter of the recess.

The dimension and arrangements of the components of a device of the invention depends upon the analysis to which the device is to be directed.

For the analysis of a single cellular component (i.e. a single mRNA transcript or a single protein) from a plurality of individual particles such as cells, a preferred device comprises a plurality of wells on a first surface of the capture support with diameter and depth of less than twice the diameter of the cell type under analysis, and a patch of analytical component (for example a capture probe) on the lid which is significantly larger than the well footprint. This relative arrangement is shown in FIG. 7. The same cellular component can thereby be easily probed in multiple cells. For example, over 10,000 wells of 20 μm diameter, spaced 30 μm apart, centre-to-centre, and arranged in a rectangular array, can be laid over an analytical component patch of 3 mm diameter. Higher densities can be achieved using alternative arrangements of wells, for example a hexagonal array, which results from the optimal packing of circles.

For the analysis of multiple cellular components from individual particles such as cells, for example a number of different mRNA transcripts, or both mRNAs and proteins, a different relative arrangement of chambers may be employed. Here, the patches of analytical component are significantly smaller than the footprint of the capture support well. Particularly preferred in this embodiment of the invention are wells surrounded by a recess, which permits the cellular components in the lysate to diffuse over a much larger area during analysis. This permits a large number of cellular components to be analysed from a single cell, as a similarly large number of analytical components can be located on an area of the lid enclosed within the chamber. For example, over 10,000 features of capture probes of 5 μm diameter, spaced 7.5 μm apart, centre-to-centre, and arranged within a rectangular array, can be overlaid by a well surrounded by a circular recess of 500 μm diameter. The relative dimensions employed in this embodiment thus mean that the lysate from a single cell can be applied to a number of analytical reagents. This arrangement is shown schematically in FIG. 8. Whilst FIG. 8 shows a rectangular grid of analytical components, other patterns can be used to achieve higher densities. In this embodiment, all analytical components within the footprint of a well may be the same as that in the other wells. In an alternative, there may be differences in the analytical components.

Chambers

The chambers formed by the joining of the capture support and the lid may have a variety of shapes. The shape of the chamber primarily depends on the shape of the well cast into the capture support. A chamber is formed from a well and the surface of the lid. The area on the surface of the lid which is incorporated into the chamber is determined by the size (cross-sectional area) of the open end of the well, including a recess if one surrounds the open end of the well.

A chamber formed from the capture support and the lid should not permit a particle, such as cell, organelle, virus or bead, therein to exit. Further the chamber should be substantially impermeable to the components released into the chamber.

Channels

In an alternative embodiment for analysis of multiple biopolymers, the chamber formed by the joining of the capture support and the lid may be a channel (FIGS. 9 & 10). In this embodiment, the first surface of the capture support comprises at least one well intersected by, or connected to, an open channel, which is sealed when joined to the lid. In order to ensure that particles are not captured in the channel during capture, the channel should be designed such that its width and/or depth are less than the diameter of the particle being analysed. In this embodiment, individual particles are captured in a well, wherein the well is intersected by or connected to the channel, once the chamber has been formed by joining of the capture support and the lid. The well may comprise part of the channel, and may be located at any point along the length of the channel, for example at either terminus of the channel, or, preferably, at any point between the termini of the channel. In an alternative, the well may be to the side of the channel. Preferably the well is located between the termini of the channel. The side of the channel formed from the lid may contain analytical components appropriate to the desired analysis. The biopolymer, for example those released following lysis of a cell, may be transported along the channel(s).

In a device containing channels, the dimensions of analysis channels can have an important impact on a device's performance. The dimensions are important not only for preventing entry of the particle, but also to reduce the distance through which molecules released from the particle must diffuse to meet analysis components within the channels.

The channels may be substantially identical to each other (e.g. in terms of dimensions, material, analytical component(s), etc.) such that, during use, cells in different channels are separately subjected to substantially the same treatment and analysis as each other, allowing direct comparison of results. The channels may be parallel to each other. In an alternative arrangement, however, channels may radiate from a central point. It is also possible to arrange parallel channels extending in different directions from a central point. An arrangement where channels run in the same direction is preferred, however, as electrokinetic movement of material is then easier to achieve.

If electroosmosis is used to move material along a channel then parts of the channel will have an appropriately charged surfaces during use. The polarity and magnitude of the charge can be selected depending on the direction and rate of movement desired in any particular analysis. Polarity can depend on both the underlying material used to make the channel, on any surface-attached material (e.g. immobilised nucleic acids), on any surface modifications and on the biopolymer of interest. The skilled person can choose these conditions according to their needs, and suitable conditions can be determined empirically.

In a device with a plurality of channels for analysing a plurality of particles, it is preferred to have channels that are substantially identical to each other (e.g. in terms of dimensions, material, immobilised reagent(s), etc.) such that, during use, particles are subjected to substantially the same treatment and analysis, allowing direct comparison of results. It is preferred that all analysis channels are substantially identical.

In some embodiments of the invention, each chamber may contain two, or more, channels extending from the well. Further, a channel can branch into two or more sub-channels. Contents may pass into each channel or sub-channel. Each channel or sub-channel can be arranged to receive substantially the same materials as the others, or different cell contents can be directed down different branches e.g. mRNA down one branch and DNA down another, or positively charged proteins down one branch and negatively charged proteins down the other.

Also preferred are serpentine channels, which provide a further way of enhancing the chances of interaction between a biopolymer and a capture reagent. Here, in a single passage through the channel, the biopolymer can provide multiple passages over a given capture reagent. The advantages of serpentine channel are discussed in reference 11.

A range of other channel arrangements are disclosed in references 8 and 11. Further variations will be evident.

Movement of Liquid Through the Support

A liquid, for example a solvent may travel through the support through a variety of means. Movement may be passive, for example by diffusion, osmosis, capillary action, or due to the effect of gravity. Wicking may be used to draw water through the support from a reservoir. In an alternative, the movement may be active, for example by pumping, or by electrokinetic means.

The solvent may contain low molecular weight reagents in solution, for example lysis, treatment and/or analytical reagents. Movement of the solvent through the support should transport these reagents as the liquid flows. If the reagents carry a charge then they may be moved through the support by the application of an electrical field. Such a field my come from electrodes which are positioned near to the capture support, for example the gold plated slides shown in FIG. 13 may be used as electrodes to drive the movement of reagents, particles (e.g. cells) or cellular components.

The Lid

A lid may perform a number of functions in a device of the invention. It may function to merely seal the one or more wells on the capture support, it may receive the biopolymers for downstream processing, or may serve analyse biopolymers. Typically, at least a portion of the lid is coated with analytical reagents

The primary function of the lid is to effectively seal the wells, to form chambers. In doing so, the lid can receive the contents of the wells while maintaining the spatial arrangement and separation of the wells, and the contents of the wells. In a simple form the lid may be a glass slide or plastic membrane. In an alternative, the lid may be functionalised. Preferably the lid is a microarray slide. Microarray slides may take a variety of forms, which are well known in the art.

The choice of materials for the lid is influenced by a number of design considerations, and suitable materials can readily be selected by the skilled person based on the requirements of a particular device. For example, the material(s) should be amenable to microfabrication, stable to the reagents used in cellular manipulation analysis, and compatible with the methods used for observing and measuring cells and molecules. Materials impermeable to the reagents used during lysis and analysis will generally be used for the lid. For some applications, it will be necessary to attach reagents covalently to the surface of a material. For some applications it will be desirable to use a hard material; other applications may need a flexible material. Where fluorescence is used for detection the material should have low intrinsic fluorescence at the excitation and emission wavelengths of the fluorophore employed. Where fluorescence is used for detection in situ in an assembled device then the material should also be transparent to the excitation and emission wavelengths of the fluorophore employed. Similar criteria apply where chemiluminescence is employed. Where electroosmosis is used to move material about the device then the material should be charged during use, or should be able to carry charge. For example, the skilled person can choose to give a positive or negative charge to silicon, glass and PDMS surfaces by derivatising them with appropriate silanising reagents. Materials that can propagate an illuminating evanescent wave (by total internal reflection) may be preferred for use with certain detection techniques.

Suitable materials and fabrication for the lid are well known. Hard materials such as silicon and glass, for which microfabrication methods have been in use for many years, can be used. Thus the lid of devices of the invention can be made from a variety of materials, including but not limited to silicon oxides, polymers, ceramics, metals, etc. and mixtures thereof. Specific materials that can be used include, but are not limited to: glass; polyethylene; PDMS; polypropylene; and silicon. PDMS is a particularly useful material, and the devices can be conveniently made by using casting, injection moulding or UV-patterning and curing.

In an alternative, the lid may be a thin flexible layer, for example a plastic membrane, such as made from LDPE or PVdC, or a nitrocellulose membrane.

In one embodiment the lid may be a gold-coated slide. Here thiol-modified analytical components can be attached to the surface of the lid, while the gold coating permits the analyser to function as an electrode.

In another embodiment, the lid may be an Indium Tin Oxide (ITO) coated slide.

In some embodiments, the lid may also incorporate recesses where analytical reagents are located. In use, the lid is used to seal the well, such that the contents released upon lysis of the particle trapped in the well are received by the recess of the lid. This provides an alternative arrangement of the lid and capture support which permits location of multiple analytical components within the chamber. The chambers of these embodiments, while formed from differently shaped lid and capture support components, possess the features and advantages as detailed above with regard to chambers formed from wells which are surrounded by a recess.

The degree of precision required in the alignment of the lid and capture support depends on both the size and the density of packing of the wells on the capture support and the recesses on the lid. Preferably, the edge-to-edge separation of the recesses on the lid is greater than the diameter of the wells on the capture support, unless highly precise alignment is being performed to ensure the wells overlie the recesses. Arrays of wells and recesses with lower density require less precision in their alignment than arrays of higher densities, because there is less chance for misalignment to occur. Similarly, those embodiments which contain recesses with diameter which is multiple times the diameter of the well, for example 5×, 10×, 15, 20×, 25×, require a lower degree of precision in their alignment than those embodiments with lower ratios of recess diameter:well diameter.

Analytical Components within the Chambers

The chambers in the device are for the segregation of biopolymers, and may include analytical components that can interact with the biopolymers to give analytical results. The analytical components in any given device will generally be chosen based on knowledge of the particle of interest in order to give analytical data of interest.

Typical analytical components that can be situated within a chamber include, but are not limited to, immobilised binding reagents. Reagents that have been used in chemical cytometry [2] can also be included. Preferred analytical components are immobilised binding reagents, such as nucleic acids for hybridisation, antibodies for antigen binding, antigens for antibody binding, lectins for capturing sugars and/or glycoproteins, etc. Preferred binding reagents are specific for a chosen target e.g. a nucleic acid sequence for specifically hybridising to a target of interest, an antibody for specifically binding a target antigen of interest. The degree of specificity can vary according to the needs of an individual experiment e.g. in some experiments it may be desirable to capture a target with nucleotide mismatch(es) relative to an immobilised sequence, but other experiments may require absolute stringency.

Different immobilised binding reagents are preferably arranged in discrete patches, to facilitate data analysis.

Chambers may include a one or more different immobilised nucleic acids for hybridising to specific nucleic acids released from the captured particles. The sequence of the nucleic acids will be chosen according to the targets of interest. Preferably, the analytical components retain specific mRNA transcripts. The immobilised nucleic acids are preferably DNA, are preferably single-stranded, and are preferably oligonucleotides (e.g. shorter than 200 nucleotides, <150 nt, <100 nt, <50 nt, or shorter). Retention of mRNA rather than DNA can conveniently be achieved by removing DNA before analysis, for example by including a DNase in the buffer used to wash excess particles from the capture support.

Other chambers include a set of different immobilised reagents for capturing proteins. These will typically be immunochemical reagents, such as antibodies, although other specific binding reagents can also be used e.g. receptors for capturing protein ligands and vice versa. Techniques for the specific capture of proteins by immobilising reagents to solid surfaces are well known in the art e.g. from ELISA, surface plasmon resonance, protein arrays, antibody arrays, etc. Antibody arrays for analysing blood (e.g. by specific capture and analysis of cytokines and intracellular signalling proteins) are already available [12] (e.g. the TranSignal™ Cytokine Antibody Arrays from Panomics [13]), and electrochemical enzyme immunoassays based on immobilised capture antibodies have been reported with a sensitivity of 10 pg/ml [14]. To detect binding in an immunochemical assay format then it is typically necessary to use a second antibody (a ‘sandwich’ assay).

A single chamber can include reagents for analysing both nucleic acids and proteins.

In a preferred arrangement the analytical components of the device are located on the lid.

In another arrangement, the analytical components of the device are located on the capture support. In a further arrangement, analytical components of the device are located on both the lid and the capture support. In the latter, it is preferred that the analytical components for analysing similar cellular components are located on the same member of the device. For example, in a device to analyse both proteins and mRNA, the analytical reagents for analysing mRNA are on the lid, and the analytical reagents for analysing proteins are on the capture support. Other arrangements, for example for analysis of DNA or polysaccharides will be evident.

The analytical reagents may also be incorporated onto analytical beads which have been captured by the support as the same time as the particles of interest. The analytical beads may then be removed following disassembly of the device and subjected to downstream analysis.

Methods for immobilising analytical reagents onto surfaces are well known in the art. Methods for attaching nucleic acids to surfaces in a hybridisable format are known from the microarray field e.g. attachment via linkers, to a matrix on the surface, to a gel on the surface, etc. The best-known method is the photolithographic masking method used by Affymetrix for in situ synthesis of nucleotide probes on a glass surface, but electrochemical in situ synthesis methods are also known, as are inkjet deposition methods. Reference 15 provides a good review of current methods, and also experimental designs, which are appropriate to the present invention. Methods for attaching proteins (particularly antibodies) to surfaces are similarly known. These methods have been applied at the scale appropriate for single cell analysis.

Immobilised nucleic acids can be pre-synthesised and then attached to a surface, or can be synthesised in situ on a surface by delivering precursors to a growing nucleic acid chain. Either of these methods can be used according to the invention.

Devices preferably contain at least 10^{N} different analytical reagents, wherein N is selected from 0, 1, 2, 3, 4, 5, 6, 7, 8 or more. Immobilisation of at least 10⁶ different oligonucleotides onto a single surface is well known in the field of microarrays. The 10^{N} different reagents will typically be arranged in 10^{N} different patches.

Each patch of immobilised reagent preferably has an area of less than 10^{X} m², where X is selected from −5, −6, −7, −8, −9, −10, −11, −12, etc. Microarrays with patch sizes in the order of 10 μm×10 μm (i.e. 10⁻¹⁰ m²) are readily prepared using current technology. Patches with a small area improve the sensitivity of detection. When materials bind to the immobilised analysis reagent they are confined to a small area, increasing signal to noise ratio.

The centre-to-centre separation of patches is preferably less than 10^{Y}m, where Y is selected from −2, −3, −4, −5, etc. Adjacent patches may abut or may overlap, but it is preferred that adjacent patches are separated by a gap. Overlapping patches are not preferred.

In a device with a plurality of chambers for analysing a plurality of particles, it is preferred that the selection, series and amount of immobilised reagent(s) within each chamber should be substantially identical such that, during use, each particle is subjected to substantially the same treatment and analysis, allowing direct comparison of results. Further details of this aspect of the invention are given below.

Chambers in devices of the invention may also be adapted to allow sequence determination of nucleic acids. In one embodiment the device contains a membrane placed over the well capture device which contains a nanopore. This nanopore can then be functionalised to allow determination of the sequence of the molecule by Nanopore DNA sequencing as disclosed in reference 16. By applying an electric field across the membrane and support, nucleic acids can be directed through the pores and in doing so can be sequenced according to this technique.

As mentioned above, a powerful aspect of the invention is to perform identical individual analysis in parallel on different cells, and the invention provides a device for individually analysing a plurality of cells, comprising a plurality of chambers, each of which is for receiving the contents of an individual cell, wherein each chamber contains a series of sequential analytical components along the chamber, and wherein the sequence of analytical components in one chambers is the same as in another chambers.

Thus a cell will experience a common set (e.g. A, B, C, D, E, F, G, . . . ) of analytical components regardless of which chamber it enters. This common arrangement of analytical components within multiple chambers means that each cell being analysed experiences the same analytical reagents, meaning that the results for one cell can readily and directly be compared to the results for another cell.

At least 10 (e.g. 10, 50, 100, 250, 500, 1000 or more) analysis chambers, or all of the analysis chambers, may contain a common set of analytical components.

Often, the common set of analytical components has the same composition and spatial arrangement in each of the chambers (e.g. all patches of immobilised reagent have substantially the same size, spacing, position, reagent concentration, etc. as each other). Thus the results from multiple chambers can readily be aligned with each other. For instance, in a device containing channels, if all channels are parallel straight lines, and if the first analytical components of all channels are aligned (e.g. FIG. 10), a straight line running perpendicular to the channels will cross the same analytical component in each of the channels, as discussed above. A detector running in a straight line perpendicular to and above the channels will therefore be able to scan in turn the results of the same single analytical test for each cell. It can then move along the direction of the channels to the position of the next analytical component and can repeat the straight line scan to obtain the results of the next single analytical test, etc.

Although each channel may have a common set of analytical components, this does not mean that all of the contents of each chamber must be identical. For instance, two chambers might have different components for detecting differing mRNAs, but the same components for detecting proteins, or indeed a common set of components for detecting certain mRNAs, such as common transcripts which are used as internal controls, but then further sets of analytical components for detecting differing RNAs.

A common set of immobilised binding reagents is preferred.

If a device includes channels, branching from the site of cell lysis, that are designed to receive different types of material (e.g. one branch for protein, one branch for mRNA which can be easily separated at low pHs based on their nascent charges) then a common series in a branched region will generally apply to only one branch e.g. all of the protein channels have a common series, but the same common series is not seen in the mRNA channels, which will have their own common series. The advantage of linear scanning parallel to the channels is still manifested in the branched arrangement.

The Device

A device according to the invention is formed from a capture support and a lid. The lid abuts the surface of the capture support comprising the wells, sealing the wells and forming chambers.

In one embodiment the device optionally further comprises an intervening membrane layer. Here, the membrane is placed over the wells prior to the application of the lid. The membrane may be selectively permeable, and so can be adapted to allow only certain biopolymers to interact with any analytical components on the lid. The membrane may serve only to capture any biopolymers, which may then be analysed downstream following disassembly of the device. The membrane may also be coated with treatment reagents, for example lyophilised enzymes or reagent containing liposomes, which are activated when the membrane is applied to the well. Suitable reagents are discussed in more detail below. The membrane may also be functionalised with analytical reagents, which may then be analysed by standard techniques in downstream processes. In some cases the membrane may have small pores through it, for example the Whatman nucleopore membrane.

In addition those features detailed above, other features of devices can include:
A reservoir. A reservoir is used to hold solutions of reagents for use with the device. The reservoir may be separate or integral to the capture support. If the reservoir is integral, it may be conveniently prepared by casting. A reservoir in a capture support is shown in FIG. 2.
One or more electrodes. Electrodes can be used to generate an electrical potential across a device, and in particular along an analysis channel e.g. to move cells by electrokinesis, electrophoresis, to allow electroporation, etc. As an alternative, the device can include contacts for the connection of external electrodes.
A light source e.g. a laser. This can be used for various purposes e.g. for cell lysis, to view the progression of a meniscus in channels, to excite fluorophores, etc.
An image capturing element, such as a camera. This may capture still and/or moving images. It will typically be a digital camera. In alternative forms, the camera may involve single point detection with a stage, or a line camera. The camera provides a means to check for the occupancy of the cell trapping sites, and also multiple occupancy of wells (e.g. by small cells in a mixed population) which can be excluded from downstream analyses if in accordance with the experimental design.
A detector, e.g. a mass spectrometer

Releasing the Contents of a Cell

When a cell has been captured, its contents can be released e.g. by cell lysis. The contents can be released in various ways. For instance, a lysis solution can be applied to the device, and a cell will be lysed in situ within a chamber. In some embodiments, this lysis solution may contain be an aqueous solution of detergent, for example SDS. In this case, the detergent should be at a concentration less than that at which it forms micelles in the aqueous solution, because the micelles will not be able to travel through the capture support. As an alternative, the chamber can be adapted to mechanically rupture a captured cell e.g. using the ‘nanoscale barbs’ described in reference 17. To ensure lysis, it is the device may be agitated so that the cell membrane is forced onto the ‘nanoscale barb’, but the agitation should not be such that the components of the device become separated. As a further alternative, the cell contents can be removed by electroporation and, depending on the magnitude of the electric field used for electroporation, a membrane may simply be opened, allowing access to a cell's contents, or may rupture, leading to cell lysis [18]. A field strong enough to cause lysis is preferred. An AC or DC electric field may be used. Alternatively an electric field may be applied in order to alter the pH of the local region of the support where the wells are located [19]. Ultrasonic vibration can also be applied to the device in order to lyse cells, as can laser light, which has previously been used to lyse single cells, as in reference 20. Lysis of single cells in a microfluidic device by osmotic shock is reported in reference 21. In an alternative method, lysis reagents may be placed into synthetic liposomes, which are already in the well when the well is sealed. This may be accomplished by a range of means, for example by immobilising the liposome onto the well or by including liposomes in buffers applied to the wells, before sealing. The liposome should be capable of being lysed to release its contents upon demand. One mechanism for doing so is through the incorporation of dyes into the liposome, which causes the liposome to lyse upon illumination with UV light, which is reported in reference 22. Similar methods may be employed for whole cells, whereby the cells are soaked in a lysis agent which is photoactivated, such that the reagent is absorbed by or adsorbed onto the cells, and the reagent activated by illumination with UV light causing lysis of the wells. Also applicable are methods of electrochemical lysis.

As an alternative, the cell contents can be released by thermal lysis. The elevated temperature required for lysis, which will depend on the specific cell type analysed, can be brought about by incubation of the device in an oven, or placing the device on a hot plate. A further alternative means of providing any heat necessary for lysis is to coat the reverse side of the lid on which the analytical components are arrayed with a layer of a conductor, such as Indium Tin Oxide (ITO), see FIG. 11. The layer of ITO is a poor electrical conductor, and so passage of a direct current through the layer results in significant energy loss, in the form of heat. This property can be exploited to controllably provide heat for the lysis and hybridisation steps. This layer may also be employed to provide a heat shock to the cells, should this be necessary, as part of the analytical protocol, or as part of an experimental procedure prior to analysis. Should temperature based lysis methods be utilised, the upper temperature used should be appropriate to the material utilised to manufacture the capture support, so that the structural integrity of this support is not lost whereupon the cell lysates from different chambers would be allowed to mix. A further advantage of using temperature as a means of cell lysis is that it permits the decoupling of the lysis and the hybridisation steps. By lysing at, and then maintaining, a temperature higher than that at which hybridisation may occur, diffusion of cellular components through the chamber may be increased. After the time deemed necessary for diffusion to occur, the temperature may be decreased to that which is suitable for hybridisation of the cellular components and the analytical components of a device according to the invention.

In a further method, for some mammalian cells, lysis may be induced using a solution which is hypotonic to the cellular cytoplasm, such that water flows into the cell to due to osmosis, and swells the cell. This swelling ultimately results in rupture of the cell membrane and the release of the lysate without any requirement for additional reagents.

Typical lysis solutions that can be used may comprise components such as: a surfactant e.g. an ionic detergent such as SDS when analysing nucleic acids, or a non-ionic detergent such as Triton-X100 when analysing proteins; a compound to digest proteins; a compound to digest nucleic acids; a chaotrope to inactivate enzymes and solubilise cellular components e.g. a guanidine salt, such as guanidinium isothiocyanate; etc. Such reagents are commonly used in existing techniques for bulk cell lysis. The choice of reagent(s) will depend on the nature of the analytes of interest. The lysis reagent should be able to travel through the capture support.

Solutions for lysis can be applied to a reservoir cast in the upper capture support (see step (f) of FIG. 2). As an alternative, a slab manufactured from the same, or alternatively a different, material as the capture support which has been previously soaked in the lysis solution may be placed upon the capture support. Instead of soaking in the lysis solution, the lysis reagent may be incorporated into the slab, for example during polymerisation. The lysis reagent is then allowed to diffuse from the slab, though the capture support and to the captured cells whereupon they are lysed.

Similar methods may be employed for the lysis of organelles.

Treatment of Cellular Components

Treatment reagents may be applied to the same area as the initial lysis solution. As with the lysis reagent(s), treatment reagents introduced in this manner should have physical properties which permit their travel through the capture support.

Instead of being applied to a surface of the support, the reagents for treatment may be present in the cell suspension initially applied to the inverted capture support, in the solutions used to wash off excess cells, or may be applied following the wash. PBS, or other suitable buffers, may be supplemented with reagents, for example DNase or proteases, such as Pronase, such that when the chambers are formed by the joining of the capture support and the lid, the treatment reagent is also present in the chamber, whereupon in can act following cell lysis. The same procedure may be applied to introduce a blocking agent, for example BSA, to the chambers to reduce non-specific/background signals.

In another embodiment the treatment reagents are in a liposome, which is lysed by laser light to release its contents, as detailed above. Further, by the incorporation of dyes sensitive to light of different frequencies into different liposomes, it is possible to lyse the sets of liposomes at different points in an experiment. One set of liposomes could contain lysis reagents, one could contain treatment reagents, another analytical reagents etc. which permits release of their contents at controlled times.

Analysing Results

The detection methods used to analyse results depend on the nature of the molecular targets and on any label that may be used. They may also depend on the strength of the signal at a given analysis site, as explained in more detail below. Quantitative detection methods are preferred.

Detection may occur in situ within the device or may occur in a disassembled device. For instance, in a device comprising a capture support and a lid, with capture reagents immobilised on the lid, the capture support can be removed after biopolymers have been captured, and the lid can be analysed separately e.g. using the reagents, techniques, devices and software already used to analyse microarrays.

For the preferred analyses (RNA and protein), further biochemical processing may be needed in order to introduce detectable labels after a target biopolymer has interacted with an immobilised binding reagent. Fluorescent labels are preferred for use with the invention.

Fluorescence in the chambers can be excited using an evanescent wave. Other sources of light for excitation can also be used e.g. lasers, lamps, LEDs, etc.

Proteins can be detected by one of several known methods that exploit antibodies. For example, a protein that has been captured by an immobilised antibody can be detected by applying a second labelled antibody specific for a different epitope from the first antibody, to form a ‘sandwich’ complex.

Any fluorescence which is detected preferably results from specific binding of two biological molecules e.g. two nucleic acids, an antibody & antigen, etc.

In situ detection of mRNAs can be accomplished using a number of methods. For example fluorophore-quencher pairs may be used. In this method, immobilised nucleic acids will incorporate a fluorophore and a quencher, which are in close proximity in the native state of the immobilised nucleic acid, in the same mechanism employed by TaqMan and Scorpion probes in standard PCR methodologies. Upon binding of a cellular mRNA, the pair is spatially separated, thus allowing detection of the binding interaction. A description of the use of such methods is given in reference 23. As an alternative, a fluorophore which changes emission or excitation frequency, depending on its chemical environment, may be used to detect immobilised nucleic acids which are or are not bound by a cellular mRNA.

High-sensitivity methods of quantitative analysis of fluorophores in a single cell analyser are disclosed in reference 8.

Sensitive detection means are provided, but a target can be detected only if it has been captured. One aim of the invention is to capture as many target molecules (i.e. the analytes for which analytical components are provided in a channel) as possible, preferably at least 50% (e.g. ≧60%, ≧70%, ≧80%, ≧90%, ≧95%, ≧99%, or even 100%) of the mRNA targets within a cell, and typically substantially all of a particular target transcript. This is particularly important for rare transcripts. This aim has implications for various features of the device and its use e.g. the size of a capture patch, the density of nucleic acids within a patch, the dimensions of an analytical chamber or channel, etc.

Analysis of the nucleic acids may also be accomplished by Transcription Mediated Amplification (TMA), which permits isothermal amplification of nucleic acids (see ref. 24).

Moving Cell Contents Through the Device

Various techniques can be used to move a biopolymers within the device, such as along a channel e.g. based on pumping, suction, electrokinesis, etc. Preferred techniques move the biopolymers electrokinetically (e.g. by electroosmosis or by electrophoresis) and require a potential to be applied across the channel, with the polarity dictating the direction of movement. Electrokinetic movement in microfabricated devices is reviewed in reference 25. When electrophoresis is used within the context of this invention, it will usually be for moving material through the device rather than for separating molecules from each other based on their mobility.

Further details of the use of electrophoretic and electrokinetic movement of materials are provided in reference 8.

Cells to be Analysed

The invention is suitable for the analysis of various cells, including both eukaryotic cells and prokaryotic cells. The invention is particularly suitable for analysing a plurality of cells which, although of the same type, are asynchronous i.e. at different stages of the cell cycle. The invention is also applicable in analyses of how individual cells in a population react to a stimulus, such as a xenobiotic, or a chemokine.

The invention can be used to analyse prokaryotic cells, such as bacteria, including, but not limited to: E. coli; B. subtilis; N. meningitidis; N. gonorrhoeae; S. pneumoniae; S. mutans; S. agalactiae; S. pyogenes; S. aureus, P. aeruginosa; H. pylori; M. catarrhalis; H. influenzae; B. pertussis; C. diphtheriae; C. tetani; etc.

Within the eukaryotes, the invention can be used to analyse animal cells, plant cells, fungi cells (particularly yeasts), etc. Preferred animal cells of interest are mammalian cells. Preferred mammals are include guinea pigs, cats, dogs, mice, rats, and primates, including rhesus macaques, tamarins and humans.

Specific cell types of interest, particularly for human cells, include but are not limited to: blood cells, such as lymphocytes, natural killer cells, leukocytes, neutrophils, monocytes, platelets, etc.; tumour cells, such as carcinomas, lymphomas, leukemic cells; gametes, including ova and spermatozoa; heart cells; kidney cells; pancreas cells; liver cells; brain cells; skin cells; stem cells, including adult stem cells and embryonic stem cells; etc. Cell lines can also be analysed.

As detailed above, the well should have depth and diameter of less than double the diameter of the cell type under test. Typical cell dimensions are given in the following table, with some example organelle and virus sizes for comparison:

| Cell | Dimensions | | Volume | |
|---|---|---|---|---|
| S. cerevisiae | 5 | μm | 66 | μm³ |
| S. pombe | 2 × 7 | μm | 22 | μm³ |
| Mammalian cell | 10-20 | μm | 500-4,000 | μm³ |
| Human T lymphocyte | 6-8 | μm | | |
| Neurophil | 10-12 | μm | | |
| Eosinophil | 10-12 | μm | | |
| Basophil | 12-15 | μm | | |
| Monocyte | 14-17 | μm | | |
| Erythrocyte | 6-8 | μm | | |
| Human oocyte | 100 | μm | | |
| Plant cell | 10-100 | μm | | |
| E. coli | 1 × 3 | μm | 2 | μm³ |
| Mammalian mitochondrion | 1 | μm | 0.5 | μm³ |
| Mammalian nucleus | 5-10 | μm | 66-500 | μm³ |
| Plant chloroplast | 1 × 4 | μm | 3 | μm³ |
| Bacteriophage λ | 50 | nm (head only) | 6.6 × 10⁻⁵ | μm³ |
| Ribosome | 30 | nm diameter | 1.4 × 10⁻⁵ | μm³ |
| Globular monomeric | 5 | nm diameter | 6.6 × 10⁻⁸ | μm³ |
| protein | | | | |

From a practical standpoint, it is easier to separate and capture cells which are in free suspension, such as unicellular organisms or circulating cells from animals. Often, however, the cells of interest will not naturally be separated in this way. In such cases, however, methods for preparing cell suspensions are well known from techniques applied to FACS.

The invention is used to analyse the contents of these cells. This does not mean that the invention must be used to analyse total cell contents e.g. as described above, unwanted materials can be removed prior to analysis. Nor must total cell contents be removed from the cell e.g. only particular fractions need to be removed, and only a partial extract need be taken. In general, however, the invention will involve cell lysis to release total cell contents, and analysis will be performed on at least mRNA transcripts and/or proteins from the cell.

It may be advantageous to treat a population of cells prior to introducing them to the device of the invention. For example, cells may be separated into fractions e.g. based on size, cell markers, etc. Separation can be achieved by a number of methods known in the art which are discussed in reference 8.

For certain applications, it would be advantageous to prefractionate cells according to size prior to feeding them to the apparatus. Cells can be prefractionated according to size by directing a cell suspension through a system of sieves before the buffer stream is dispensed onto the capture support. Methods for extracting single cells from larger cell masses are disclosed in reference 26.

There are a number of ways to introduce cells onto the capture support of the invention. In most cases, the cells will be suspended in a buffer solution e.g. to ensure that they retain their integrity, and a characteristic size and shape. The suspension may be applied to a receptacle that feeds into a line which spreads the cell suspension onto the capture support where the cells are captured. As an alternative, the cells may be dispensed manually, for example from a pipette.

Cells can be dispensed onto the support from other cell separating apparatuses e.g. from a cell sorter such as a MACS or FACS device, from a cell fractionation column such as those used to separate red and white blood cells, etc.

Observation of Cells

When it is desired to observe particles such as cells within a device, a microscope will usually be used. Because of the small optical contrast with respect to the buffer and typical microfluidic structures, it may be desirable to use contrast enhancement e.g. using techniques such as phase contrast microscopy, differential interference contrast microscopy, fluorescence microscopy, etc. In many cases, however, a conventional light microscope can be used.

Depending on the requirements of the experiment, the microscope may be operated manually (focus, positioning, selection of the field of view, etc.), or it can be fully automated. The images may be used for diagnostic purposes and fault detection (accidental capture of two particles (e.g. cells), capture of contaminants, etc.), as well as for documentation purposes. Image analysis may be used to distinguish between different captured cell types.

Non-Cellular Particles

As well as analysing cells, the invention can be used to analyse other particles. These particles may be natural or man-made. Thus the invention can be used to analyse single organelles in eukaryotic cells, and particularly nuclei (e.g. for transcription factors), mitochondria and plastids (e.g. chloroplasts). Organelles can be prepared from cells prior to introducing them to the device of the invention. The organelles can then be further captured and treated in the same way as described above for whole cells. The wells of the capture support will be sized accordingly.

The invention may be used for the capture of beads or synthetic liposomes or vesicles. The beads may be functionalised, and may have been previously used to capture proteins, or other molecules. Capture may then be followed by analysis of the material captured on the surface of the beads or within the liposomes. The material can analysed as discussed above for whole cells, and similarly other reagents added for use in the analysis or for treatment of the captured molecules prior to, or during, treatment.

Oil-in-water or water-in-oil emulsions are suited for analysis by the invention. The droplets in this emulsion can be individually trapped and analysed. If the droplets are synthesized so that they each contain individual DNA fragments then this can be combined with the membrane containing a pore for sequencing to make devices amenable to large-scale parallel sequencing operations.

In some embodiments, the particles captured by the device may be colloidal particles.

In some embodiments, it may be desired to capture particles of assorted types, for example a cell and one or more functionalised beads.

Further Features

Because devices of the invention have a very small scale, they can easily become blocked by contaminants such as dust. Filtration of samples prior to analysis is therefore preferred. A filter can be integral with the device of the invention or may be separate.

Once cells have been trapped, they may be examined under a microscope for features such as size and shape. For more detailed characterisation, they may be stained, for example with fluorescently labelled antibodies or alternative dyes which stain markers on the cell surface, such as lectins, before microscopic examination. Staining of the cells may be performed before or after trapping. Such information is useful for association with molecular characterisation, the main objective of the invention.

Manufacture

The capture support may be prepared by casting in a mould. This process is shown schematically in FIG. 1. In this Figure, the material is poured into the mould, and allowed to solidify. The mould is then removed to leave the capture support. The mould is produced with the inverse, or negative, of the features desired in the capture support.

An advantage of using a mould and recasting the capture support is that there is no necessity to use the same support repeatedly; the old cast can be discarded and a new one made. Therefore, in addition to the alleviation of requirements for thorough cleaning of the support between experimental runs, any possibility of cross contamination between experiments resulting from persistence of reagents in the support is removed.

Preferably the material added to the mould is a monomer, or partially polymerised monomer, where full polymerisation of the material is allowed to occur within the mould.

Moulds

In one method of generating a mould for casting of the capture support, a photoresist is spin-coated on a substrate to a thickness equal to the desired well depth (the depth will depend upon the cell type to be analysed, as will be evident to the skilled addressee). A pattern is then selectively etched away by means of photolithographic techniques. The pattern produced at the end of the photolithographic process will be the inverse, or, in other words, the negative, of the desired pattern of the well or channel part of the device. The dimension of the wells and/or channels produced by the inverse of this pattern should be chosen such that they are appropriate to the analysis performed. As with the depth of the spin-coated layer, such design is no burden to the skilled addressee. The total thickness of the capture support on which the wells are patterned should be such that the device is rigid enough to be handled without breaking, but also thin enough to allow fast permeation of lysis buffer across its depth. Typically permeation of lysis buffer will occur within 5-10 minutes. Once prepared, the mould can then be used repeatedly to cast a series of identical capture supports, optionally using a range of different materials as part of the process of experimental optimisation.

Other methods of manufacture of the mould include wet chemical etching (isotropic and anisotropic), electrochemical etching, wet photoetching, dry chemical etching, sputter etching, ion milling, reactive ion etching, deep reactive ion etching (DRIE), x-ray deep-etch lithography and electroforming (a.k.a. LIGA), vapour phase etching (with XeF2), focused ion beam milling, laser machining, ultrasonic drilling, electrical discharge machining, mechanical drilling, milling, grinding, honing, lapping, sawing; hot embossing, injection moulding, micro thermoforming and casting

In use, the mould receives the material from which the capture support is to be made, and then is incubated under conditions which allow the material to set to form the solid capture support. The conditions for incubation depend on the material used.

Method of Individual Cell Analysis Using a Capture Support

A cell is captured within a well on a first surface of the capture support when a cell suspension is dispensed onto this surface of the capture support. Multiple individual cells may be captured by a capture support comprising a multiplicity of wells on the first surface. The capturing process can be envisaged by reference to FIG. 2. The cells are then allowed to settle into the wells (step b)). Optionally, the capture support may be agitated to aid the movement of the cells into the wells. Any agitation should be only in the horizontal, x and y axes, and such that it does not cause cells which are present in a well to exit that well. The optimum level of agitation can be empirically determined for the cell type under analysis. Further, the agitation should not cause lysis of the cells. Untrapped cells can then be washed away using an excess of buffer, which may optionally contain further reagents for the treatment of the cells or cellular components. Again, care should be taken when the washing step is performed to ensure that cells which are resident in the wells are not disturbed. In capture supports where the well have been further suitably modified to contain reagents such as antibodies to aid capture of specific cells (see above for further discussion), washing may be more vigorous as the binding of the antibody to the cell surface makes increases the forces necessary to displace the cell from the well, and indeed may be necessary to displace cells of the correct size but of the wrong cell surface marker phenotype. At this point it may be preferable to monitor occupancy of the wells, for example, through the use of a microscope, digital camera and appropriate imaging software. The steps of dispensing the cell solution, optional agitation, and washing may be repeated, as necessary, until the desired proportion of wells are occupied by cells.

Optionally, the cells may be arrested, or fixed, prior, during or shortly after their capture using the cell capture device, for example using methanol. In performing this step, the importance of the time taken for the any lysis reagent to travel through the capture support is reduced because the cells are no longer metabolically active, and accordingly any enzymes capable of breaking down the biopolymers of interest are inhibited.

After the cells have been caught, the lid, as described above, is placed upon the wells to seal them (FIG. 2(c)). The device can now be manipulated such that they are in any orientation, but are preferably inverted such that the lid is now beneath the capture support (as in FIG. 2(d)).

Further, optionally, captured cells may be subjected to treatment prior to lysis. For example, the cells may be exposed to one or more stimuli, such as chemokines or xenobiotic compounds to induce a response. The cells may also be incubated in culture medium within the chambers prior to lysis. This step may follow any exposure to stimuli, or may be performed straight after capture to allow the cells to recover from the prior procedures. This medium may be present prior to capture of the cells, or may be added following capture of the cells.

Cells captured in the device are now lysed. Lysis may be by the application of a lysis solution to a second surface of the capture support. The device is then incubated under conditions that permit the liquid and any reagent it contains to travel through the support to the wells where the liquid can interact with the cells and bring about their lysis. The lysis solution is applied at a second surface of the capture support. The second surface will usually be the opposite face from the first surface e.g. where the support is cuboid (as in FIG. 2(e)). In some embodiments, however, the second surface is a portion of the first surface. For example it may be possible to apply the lysis solution around the margin of the wells after the wells have been sealed by the joining of the capture support with a lid.

As an alternative, the cells may be lysed by thermal or mechanical methods, or by electroporation, or indeed by any method described above.

After lysis, the released cellular contents are received by the lid. The device is incubated under conditions which allow the cellular components and the analytical components of the lid to interact. After this incubation, the lid may be removed and analysed by standard microarray methods relevant to the experiment being performed (FIG. 2(g)). In an alternative, some or all analysis may be performed in situ.

This method of using the capture support may be easily adapted for the capture and analysis of other particles.

Alternative Device

In the devices recited above, the capture support is permeable, and the lid is impermeable. In contrast, a device according to the invention may also be made of an impermeable capture support, and a permeable lid. The impermeable capture support may take the form of a picowell plate (for example similar to that detailed in reference 27, or the Nunc Live Cell Array). The permeable lid will typically be made from the materials detailed above for the permeable capture support.

In use, a particle is captured in the impermeable capture support, the wells in the impermeable capture support are sealed by the application of the permeable lid to form chambers. Reagents, for example lysis reagents when the particle is a cell, may then be applied to the lid which permits the reagents to travel through the lid to reach the chambers.

More generally, the invention provides a device with a permeable component and an impermeable component, wherein one component contains wells and one component has analytical reagents. In some devices both components may have analytical reagents. In those devices with analytical reagents on just one component, the wells and analytical devices are usually on different components.

General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

The use of terms such as “diameter” and “circumference” in relation to an element does not necessarily imply that the element is circular (or, in a three-dimensional context, spherical). Further, the term “diameter” in a non-circular element refers to the longest straight line distance within a cross-sectional plane, for example between the vertices of a square, or the length of an ellipse.

The term “antibody” includes any of the various natural and artificial antibodies and antibody-derived proteins which are available, and their derivatives, e.g. including without limitation polyclonal antibodies, monoclonal antibodies, chimeric antibodies, humanized antibodies, human antibodies, single-domain antibodies, whole antibodies, antibody fragments such as F(ab′)₂ and F(ab) fragments, Fv fragments (non-covalent heterodimers), single-chain antibodies such as single chain Fv molecules (scFv), minibodies, oligobodies, dimeric or trimeric antibody fragments or constructs, etc. The term “antibody” does not imply any particular origin, and includes antibodies obtained through non-conventional processes, such as phage display. Antibodies of the invention can be of any isotype (e.g. IgA, IgG, IgM i.e. an α, γ or μ heavy chain) and may have a κ or a λ light chain.

### BRIEF DESCRIPTION OF THE DRAWINGS

FIG. 1 is a schematic of the production of the cast for the capture support and casting of the capture support.

FIG. 2 is a schematic of the cell capture and lysis steps in the use of devices according to the invention.

FIG. 3 is a diagram of wells in the capture support.

FIG. 4 shows cells captured in wells in the capture support.

FIG. 5 is a diagram of wells in the capture support surrounded by a recess.

FIG. 6 shows cells captured in wells in the capture support surrounded by a recess.

FIG. 7 shows patterned wells overlaying much larger analytical component features.

FIG. 8 shows patterned wells overlaying much smaller analytical component features.

FIG. 9 is a cross-sectional view of a device for single cell capture wherein the chambers comprise channels.

FIG. 10 is a top-down view of a device for single cell capture of a plurality of single cells wherein the wells intersect channels.

FIG. 11 shows a modification to the device to include a method for localized heating of the glass slide, obviating the need for an external apparatus such as a Peltier stage or hotplate.

FIG. 12 is a graph showing the measurements of temperature Vs time at different operating voltages for an ITO coated slide.

FIG. 13 shows a modification to the device to include a method for forced ‘removal’ of biopolymers of interest trapped in the polymer/gel matrix.

FIG. 14 is a graph showing %occupancy of wells of varying well diameter by MEL c88 cells.

FIG. 15 is a graph showing the percentage of wells which are empty, singly occupied and multiply occupied by MEL c88 cells in capture supports with varying well diameter.

FIG. 16 is an image showing Cy3 fluorescence on a lid member following reverse transcription of immobilised RNA.

FIG. 17 is an image showing the outlines of Cy3 fluorescence, numbered consecutively, on a lid member following reverse transcription of immobilised RNA.

FIG. 18 is a graph showing the instensity of signal from between occupied wells and through occupied wells (18A and 18B, respectively).

FIG. 19 is an image showing the fluorescence of Wheat Germ Agglutinin (WGA) stain (FIG. 19A) and Cy3 label after reverse transcription (FIG. 19B), from wells initally occupied by WGA stained MEL c88 cells.

### MODES FOR CARRYING OUT THE INVENTION

Mould and Capture Support Manufacture

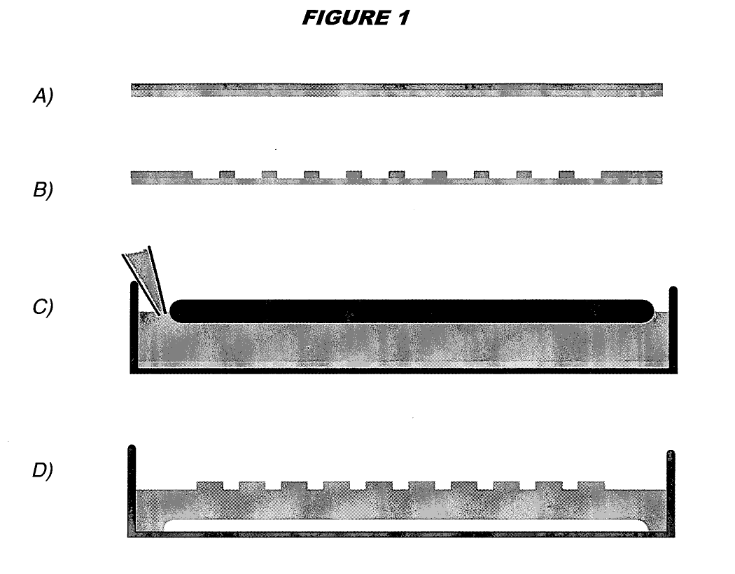

A schematic of the process for manufacturing a capture support according to the invention is shown in FIG. 1, including manufacture of the mould. In step a) of the figure, a photoresist, often SU-8, was spin coated onto a substrate layer. The depth of the photoresist was equal to the depth of the wells or channels desired in the capture support. The width of the material remaining on the base layer (step b) is the width of any well or channel in the capture support. Photolithographic techniques were then used to selectively etch a pattern of wells, and when required channels, into the photoresist. This mould was then placed into a master casting device. Material was then injected into the cast and allowed to solidify. Further optional features were also cast into the polymer using the master cast. In FIG. 1, an optional channel to act as a reservoir for lysis reagents was cast into the capture support on the opposite side of the support to the micro-structured pattern of wells. The produced capture support is shown in step d) of FIG. 1.

Polymers for Capture Support Manufacture

Typically a polyacrylamide polymer was used to manufacture the capture support. For such acrylamide gel polymers, the following recipes were used:

| | | | |
|---|---|---|---|
| Deionised RNase-free water | 15 ml | 17.5 ml | 20 ml |
| Acrylamide 40% | 7.5 ml | 5 ml | 2.5 ml |
| PBS 10X | 2.5 ml | 2.5 ml | 2.5 ml |
| TEMED | 50 μl | 50 μl | 50 μl |
| AMPS | 250 μl | 250 μl | 250 μl |

Cell Capture and Lysis

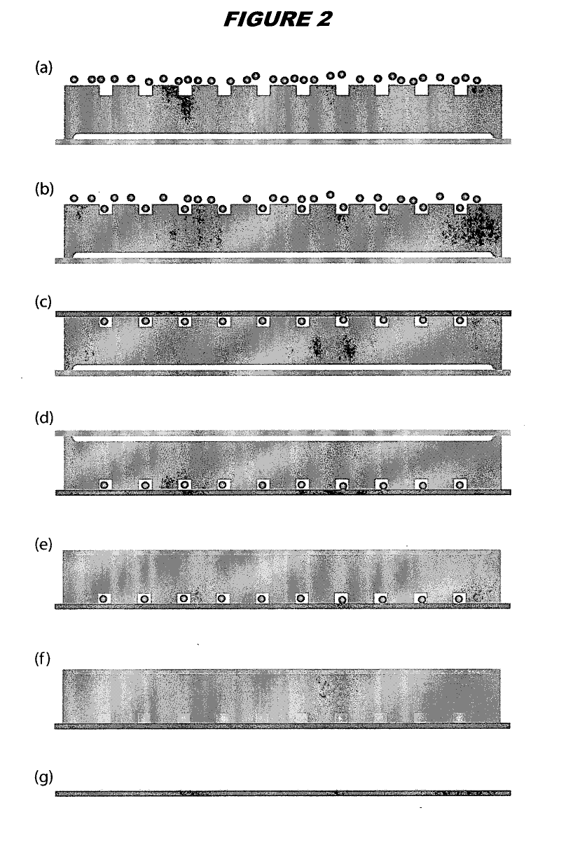

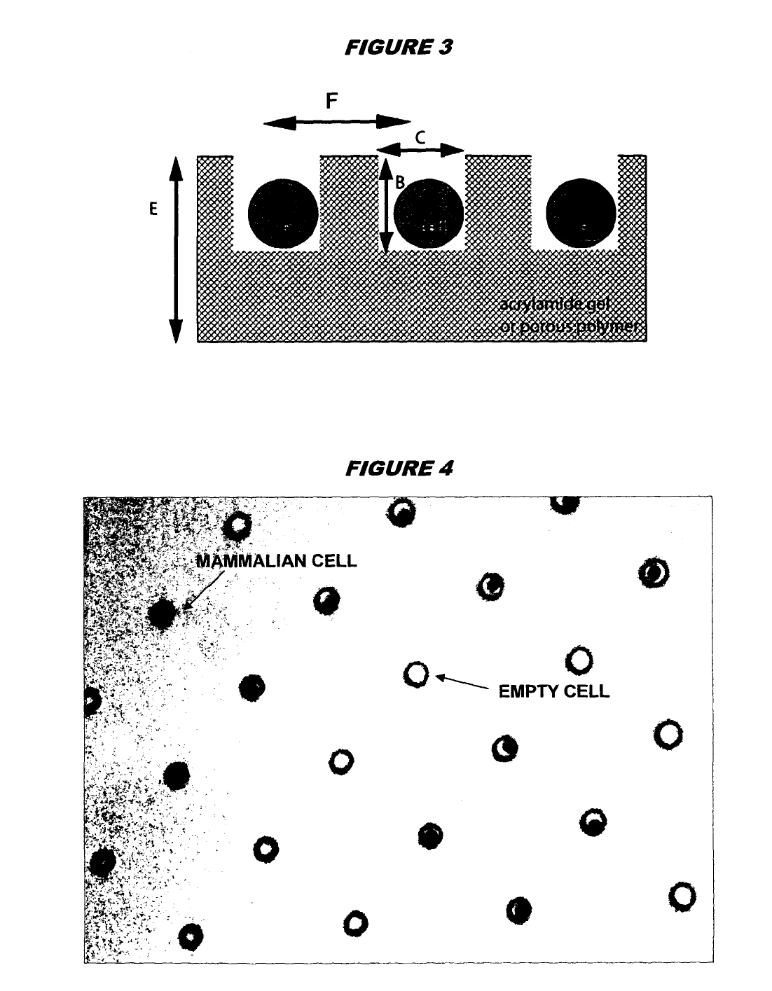

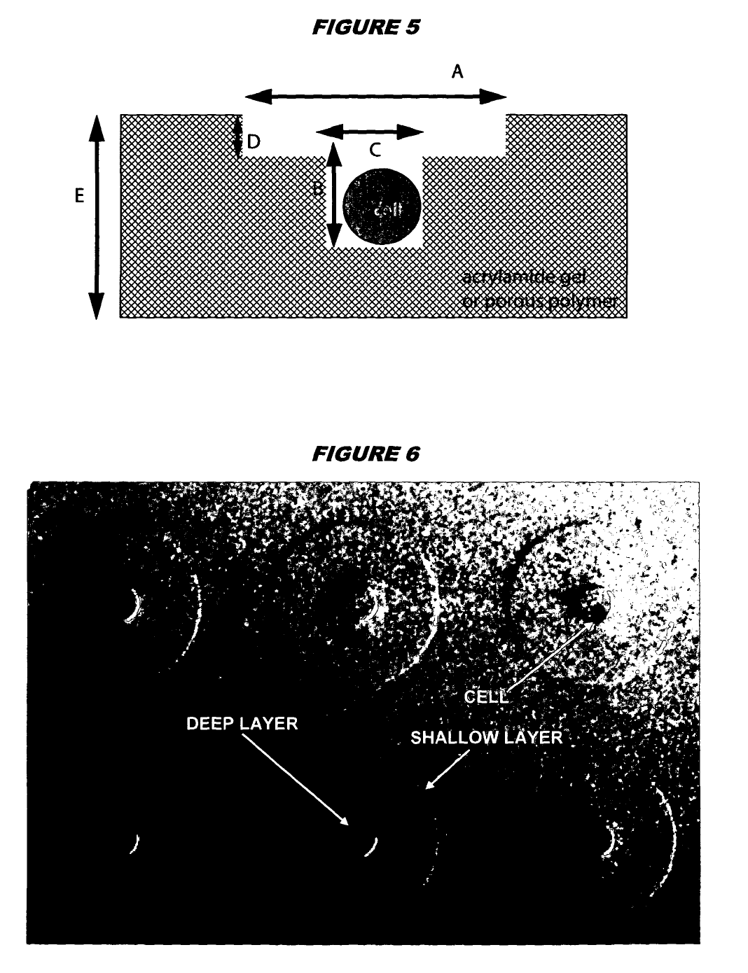

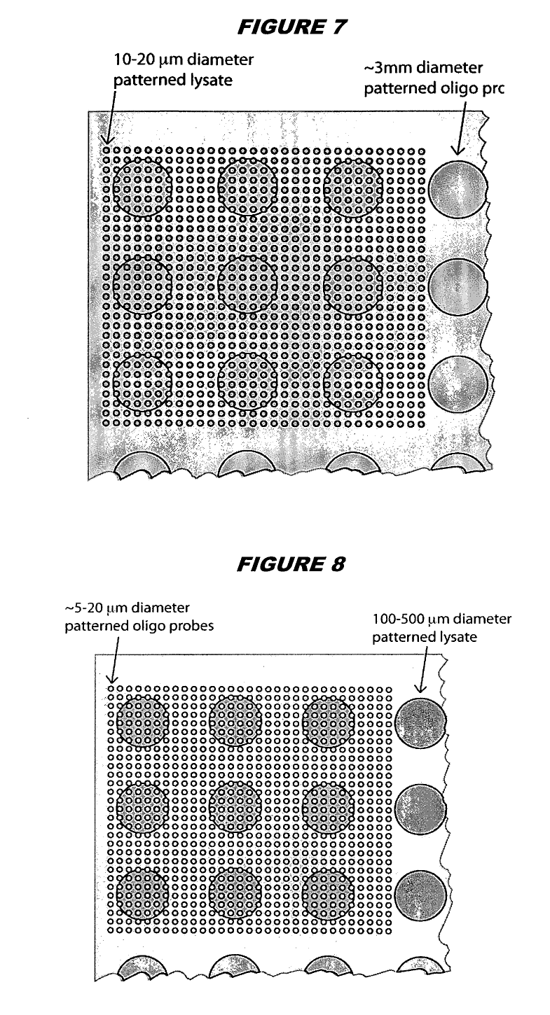

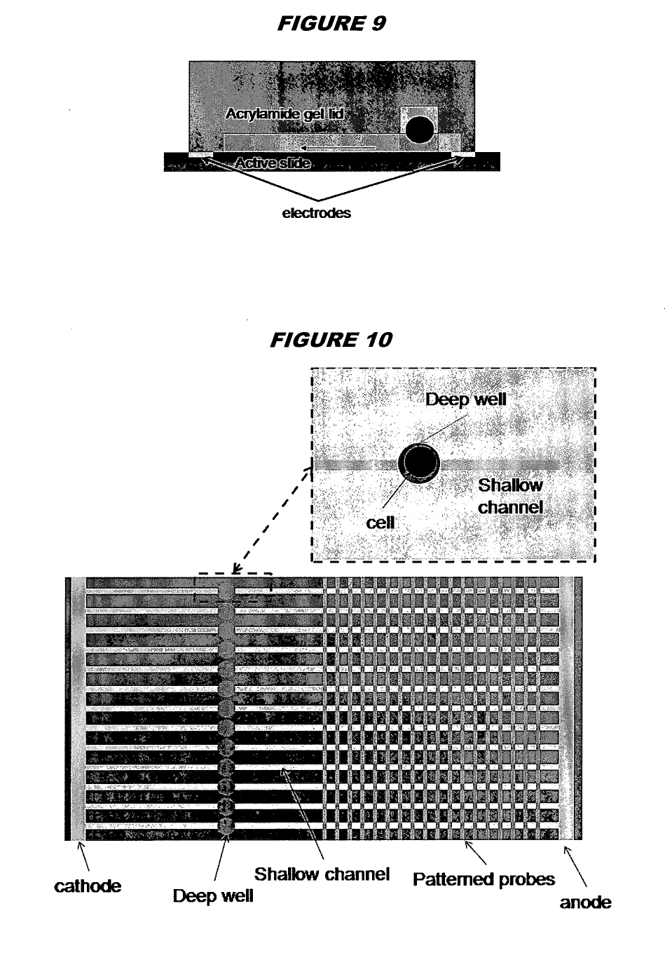

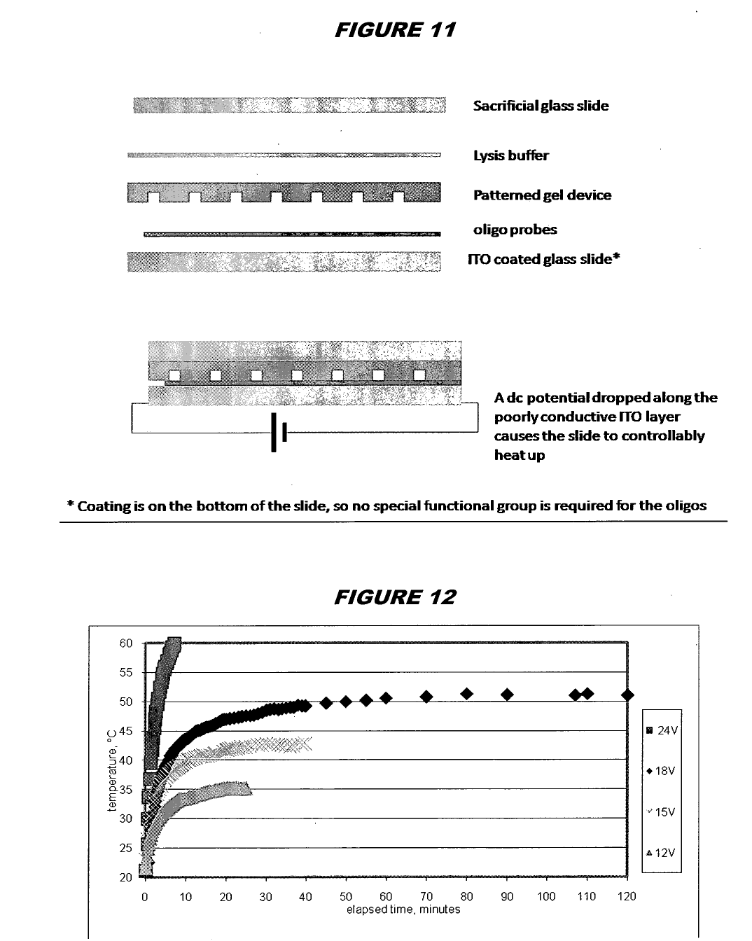

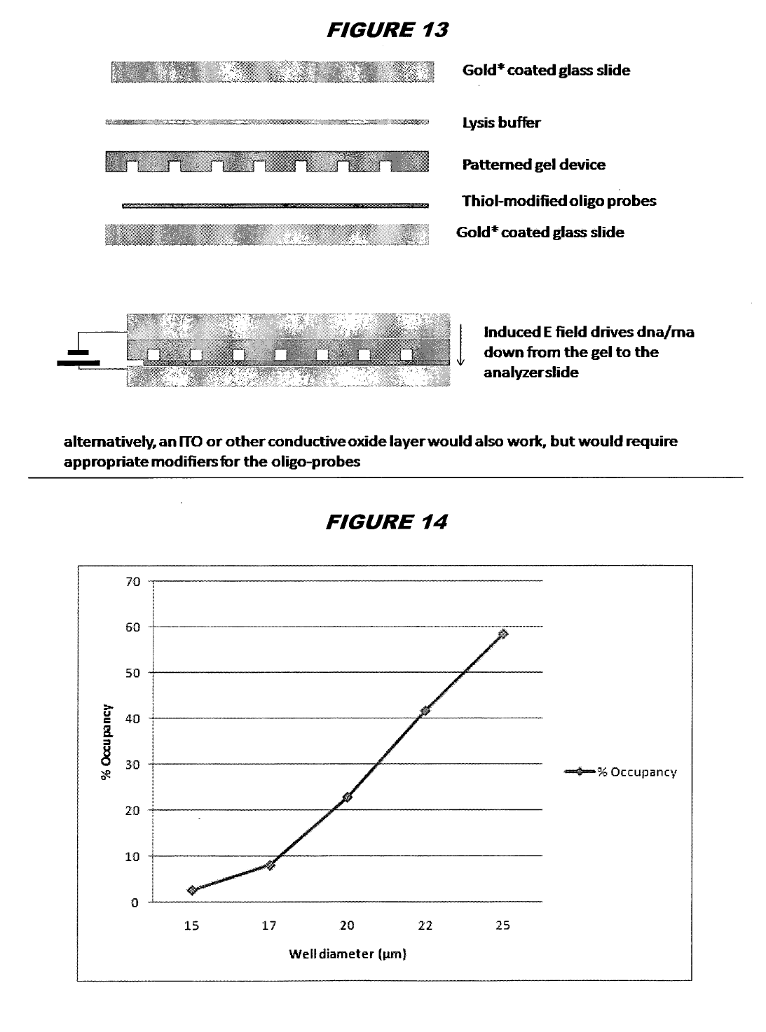

FIG. 2 shows a schematic of an operation of a device according to the invention. The capture support was placed on a substrate, in this figure a sacrificial glass slide, with the wells cast into the support facing upwards. A cell suspension was then pipetted onto the support. The cells scattered, with some cells entering the wells and some cells remaining on the surface of the capture support, between the wells. Excess cells were rinsed away with a physiologically acceptable wash buffer. Further reagents were often added to the cells at this point, for example Pronase. A lid with one or more analytical reagents was then placed onto the inverted capture support, such that the analytical components were now located within the chambers containing the cells (step c). Step d) shows inversion of the device, such that the capture support now rested on the lid. The sacrificial glass slide was removed a cell lysis solution pipetted onto the exposed reservoir on a second surface of the support. The device was then incubated to allow the perfusion of the lysis buffer through the permeable capture support. Once the lysis buffer reached the chambers containing the cells, the cells lysed (step f), whereupon hybridisation of the cellular components and analytical components occurred. Optionally, some or all of the analysis was performed in situ, while the capture support and lid were still joined. In an alternative, the capture support was removed prior to further processing of the lid, as if shown in step g) of FIG. 2. Cells captured in wells are shown in FIGS. 3 and 5. FIG. 4 shows cells captured in a well, and FIG. 6 shows cells captured in a well surrounded by a recess.

Optimising Well Dimensions

In order to ensure the maximum single occupancy of the wells in the support by a specific cell line, a series of experiments were conducted. The cell line used was MEL c88 [28], which has an approximate diameter of 15 μm, but the process is the same regardless of cell type or cell line.

5 different supports were tested, with varying well diameter and centre-to-centre distance (pitch). The dimensions were (diameter and centre-to-centre distance): 15 μm and 35 μm, 17 μm and 35 μm, 20 μm and 40 μm, 22 μm and 45 μm, and 25 μm and 40 μm.

The MEL c88 cells were at a concentration of 1×10⁶ ml⁻¹. 20 μl of cells (approx 20,000 cells) were applied to the support. The support was then washed with 50 μl aliquots of PBS until the surface of the support was clear of residual cells.

Images were then taken with a light microscope. Well occupancy was determined by automatic counting software, or manually.

Although the 25 μm diameter wells had the highest capture rate, the vast majority of wells had double occupancy (or greater). This therefore discounted this design from further use with MEL c88 cells. The support containing 22 μm diameter wells, whilst displaying some double occupancy, had a much superior capture rate than the 20 μm diameter wells (FIG. 14), even taking the double occupancy into account. The 22 μm diameter well was therefore the preferable size for using with MEL c88 cells.

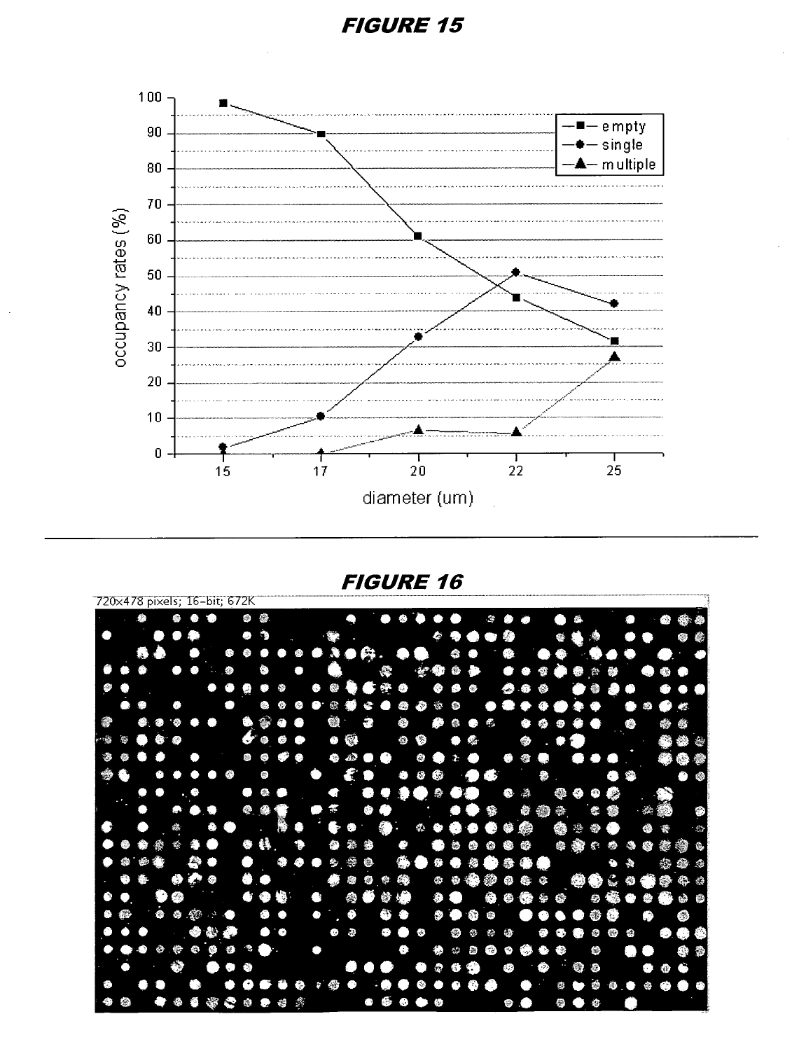

As shown in FIG. 15, for diameters smaller than 20 μm, the multiple occupancy rate for MEL c88 cells was zero, but the single occupancy rate was also relatively low (up to 10%). By increasing the well diameter, the overall occupancy rate increases monotonically. When the well diameter was 22 μm, the single occupancy rate exhibited a local maximum at ˜50%. When the diameter was 25 μm, more than a third of all occupied wells were occupied by multiple cells.

In situ Cell Lysis

For lysing cells, four buffers were used. The first lysis buffer was SDS 10% (v/v) in deionised water. The second lysis buffer was SDS 2% (v/v) in phosphate buffered saline (PBS). The third lysis buffer was SDS 2.5% (v/v) in PBS. The fourth lysis buffer was SDS 5% (v/v) in PBS.

In one experiment, MEL c88 cells were captured using capture support with 25 μm diameter and 45 μm pitch. Once the cells had been captured, the support was inverted on to a plain microscope slide and the third lysis buffer (2.5% SDS in PBS) was added. The device was viewed through a digital camera attached to an inverted microscope. 5 minutes after the addition of the lysis buffer, a first picture was taken. Subsequent pictures were then taken every 4 seconds. Total cell lysis was observed at approx 8-9 minutes following the addition of lysis buffer.

Detection of Cellular Contents Following Capture and in situ Lysis

An aliquot of 200 μl of MEL c88 cells at 2.5×10⁶ ml⁻¹ (approx. 500,000 cells) was applied to an 8% polyacrylamide support with 22 μm diameter and 50 μm centre-to-centre distance. Excess cells were washed off with repeated 200 μl aliquots of PBS. After washing 115,359 out of approx. 450,000 wells were full. 30 μl of Pronase was applied to the cells. The wells were sealed with a lid which was coated with oligo(dT)₄₀. This oligonucleotide is capable of hybridising to poly(A)-tailed RNA. Cells were lysed by addition of lysis buffer (SDS 2% (v/v) in PBS) to the support followed by incubation at 40° C. for 10 minutes. Hybridisation was then allowed to proceed for 60 minutes at 40° C. in the lysis buffer. The device was then disassembled and the lid member used in the subsequent steps. Labelled cDNA synthesis from the immobilised RNA was performed using Superscript-III (Invitrogen) and Cy3 labelled dCTP at 50° C. Unincorporated label was washed off with 10 washes at room temperature, each lasting 10 minutes. The first wash was performed with Agilent Gene Expression Wash Buffer 1. Washes 2-10 were performed with Agilent Gene Expression Wash Buffer 2.

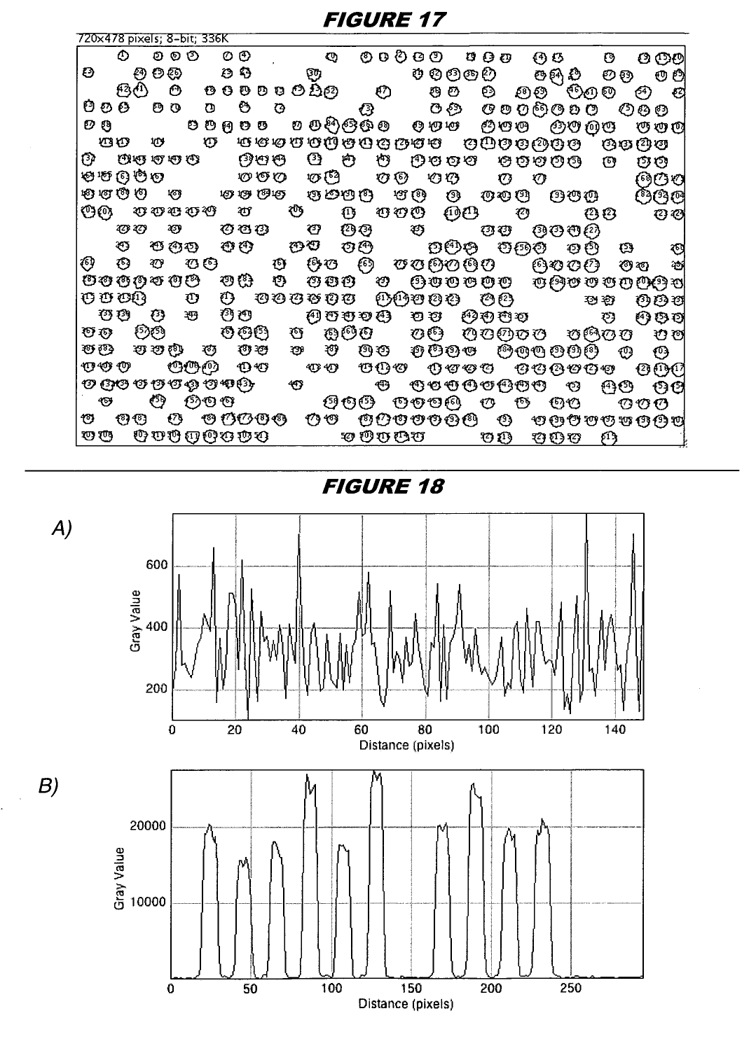

The lid member was then scanned using an Axon 4400A scanner (Molecular Devices) with 2.5 μm pixel size. The results of the scan are shown in FIGS. 16 and 17. Some of the wells were not filled, which corresponds to the “empty” spots in between visible spots. A low and consistent background was observed between spots of occupied wells, but this background was significantly lower than the signal from the occupied wells. A comparison of the background from empty wells and the signal from an occupied well is shown in FIG. 18.

Cell Surface Marker Staining

100 μl of MEL c88 cells at 1×10⁶ ml⁻¹ (stained with either 100 μg or 200 μg of Wheat Germ Agglutinin (WGA)) were applied to an 8% polyacrylamide support with 20 μm diameter and 40 μm centre-to-centre distance. Excess cells were washed off with repeated 200 μl aliquots of PBS. Pronase was applied to the cells on the support. The wells were sealed with a lid which was coated with oligo(dT)₄₀. Prior to lysis, the captured cells were observed using a fluorescence microscope. The captured cells were seen to fluoresce red—indicating that lectins were expressed extracellularly. Such staining of cell surface markers can used to detect differences in cell types when a heterogeneous cell population is added to the capture support.

Cells were lysed by addition of lysis buffer (SDS 2% (v/v) in PBS) to the support followed by incubation at 40° C. for 10 minutes. Hybridisation was then allowed to proceed for 60, minutes at 40° C. in the lysis buffer. The device was then disassembled and the lid member used in the subsequent steps. Labelled cDNA synthesis from the immobilised RNA was performed using Superscript-Ill (Invitrogen) and Cy3 labelled dCTP at 50° C. Unincorporated label was washed off with 10 washes at room temperature, each lasting 10 minutes. The first wash was performed with Agilent Gene Expression Wash Buffer 1. Washes 2-10 were performed with Agilent Gene Expression Wash Buffer 2.

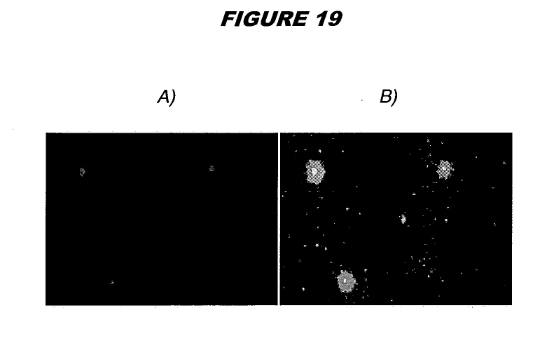

The lid member was then scanned using an Axon 4400A scanner (Molecular Devices). Following the RT reaction, barely any residual WGA dye could be observed. This demonstrated that staining of the cells with a fluorescent dye may be performed prior to RT without risk of the fluorescent dye interfering with the subsequent detection of fluorescent moieties used in subsequent steps. Residual WGA left after RT is shown in FIG. 19A. This signal is evidently much lower than the fluorescence from the Cy3 label incorporated into the cDNA by the RT step (FIG. 19B).

It will be understood that the invention has been described by way of example only and modifications may be made whilst remaining within the scope and spirit of the invention.

### REFERENCES

[1] Cossman et al. (1999) Blood 94:411-16.

[2] Krylov et al. (1999) Cytometry 37:14-20.

[3] Bao & Suresh (2003) Nature Materials 2:715-725.

[4] Andersson & van den Berg (2004) Curr Opin Biotechnol 15:44-49.

[5] U.S. Pat. No. 6,524,456.

[6] U.S. Pat. No. 6,538,810.

[7] Wu et al. (2004) PNAS USA 101:12809-13.

[8] WO2006/117541

[9] Madou (2002) Fundamentals of microfabrication: the science of miniaturization ISBN 0849308267

[10] Fahy et al. (1993) Nucleic Acids Research 21: 1819-1826.

[11] WO2008/056160

[12] Utz et al. (2005) Immunol Rev 204:264-82.

[13] Gershon (2003) Nature 424:581-7.

[14] Le et al. (2005) Front Biosci 10:1654-60.

[15] Nakaya et al. (2007) pages 265-307 of Nucleic Acid Hybridisation ISBN 1402060394

[16] Clarke et al. (2009) Nature Nanotechnology 4:265-270.

[17] Di Carlo et al. (2003) Lab on a Chip 3:287-291.

[18] Przekwas et al. (2001) pages 214-217 of Modeling and Simulation of Microsystems. ISBN 0-9708275-0-4.

[19] Nevill et al. (2007) Lab Chip 7: 1689-1695

[20] Sims et al. (1998) Anal Chem 70:4570-7.

[21] Prinz et al. (2002) Lab Chip 2:207-12.

[22] Khoobehi et al. (1990) Lasers in surgery and medicine 10: 60-5

[23] Buzdin and Lukyanov (2007) pages 85-96 of Nucleic Acid Hybridisation ISBN 1402060394

[24] Stary et al. (1998) Journal of Clinical Microbiology 36.2666-2670.

[25] Wong et al. (2004) IEEE/ASME Transact Mechatron 9:366-76.

[26] U.S. Pat. No. 6,420,105.

[27] WO2006080000

[28] Oliff et al. (1981) Blood 58: 244-254.

1. A capture support for individually capturing cells of interest, comprising a first surface including at least one well sized to accommodate an individual cell, wherein the support is made of a differentially permeable material which permits movement of a solvent and any low molecular weight species through the support from a second surface of the support to a well, but which is substantially impermeable to biopolymers.

2. A capture support for individually capturing particles, comprising a first surface including at least one well with a diameter of less than 200 μm and depth of less than 200 μM, wherein the support is made of a differentially permeable material which permits movement of a solvent and any low molecular weight species through the support from a second surface of the support to a well, but which is substantially impermeable to biopolymers.

3. The capture support of claim 1, wherein at least one of the at least one wells is surrounded by a recess.

4. The capture support according to claim 3, wherein the recess is 0.1-5 μM deep.

5. The capture support according to claim 1, wherein the low molecular weight species have MW less than 1000 Da.

6. The capture support according to claim 1, wherein at least a portion of at least one well's surface is coated with reagents to aid capture of the cells or particles.

7. The capture support according to claim 1, wherein at least a portion of at least one well's surface is coated with one or more analytical reagents which permit analysis of biopolymers.

8. The capture support according to claim 1, wherein the capture support is made from a permeable polymer.

9. The capture support according to claim 8 wherein the permeable polymer is a polyacrylamide gel.

10. The capture support according to claim 1, wherein the at least one well is intersected by, or connected to, an open channel.

11. The capture support according to claim 1, wherein the capture support comprises a reservoir integral to the capture support.

12. A device for analysing the contents of an individual cell comprising (i) a capture support according to claim 1 and (ii) a lid for receiving the contents of a well.

13. The device according to claim 12, wherein at least a portion of the' lid is coated with analytical reagents.

14. The device according to claim 13, wherein the lid comprises recesses where the analytic reagents are located.

15. A process for analysing one or more cells of interest, comprising steps of: capturing the cell(s) within well(s) of a capture support of claim 1; sealing the well(s) with a lid to form one or more chamber(s) from which captured cells cannot exit; releasing the contents of cell(s) such that they remain in the one or more chambers; allowing the released contents to interact with one or more analytical component(s) within the one or more chambers, thereby permitting analysis of the contents.

16. A mould for the manufacture of a capture support according to claim 1, wherein the mould is capable of receiving material which solidifies within the mould.

17. A method for the manufacture of a capture support according to claim 1, said method comprising the steps of: a) adding to a mould a material capable of solidifying; b) incubating the mould under conditions which permit the material to solidify; and c) separating the mould and the solidified material, to leave a capture support according to claim 1.
